(19) United States
(12) Patent Application Publication
(10) Pub. No.: US 2026/0124326 A1
(43) Pub. Date: May 7, 2026

(54) USE OF CYCLODEXTRINS AS A RADIOSTABILIZER

(71) Applicant: GE Healthcare Limited, Aberdeen (GB)

(72) Inventors: Kristine Opsvik Wikene, Aberdeen (GB); Imtiaz Ahmed Khan, Aberdeen (GB); Graeme Mcrobbie, Aberdeen (GB)

(21) Appl. No.: 19/425,391

(22) Filed: Dec. 18, 2025

Related U.S. Application Data
(63) Continuation of application No. 17/770,908, now Pat. No. 12,527,884.

(30) Foreign Application Priority Data
Oct. 21, 2019 (GB) 1915206.5

(51) Int. Cl. A61K 51/04; A61K 47/10; A61K 47/22; A61K 47/40

(52) U.S. Cl. CPC A61K 51/0459; A61K 47/10; A61K 47/22; A61K 47/40

(57) ABSTRACT

The present invention provides a radiopharmaceutical composition comprising the following four components: (i) a radio-labelled compound; (ii) ethanol; (iii) a stabilizer of the radio-labelled compound; and (iv) a cyclodextrin.

The present invention also provides a radiopharmaceutical composition comprising: (i) a radio-labelled compound; (ii) a stabilizer of the radio-labelled compound, wherein the stabilizer comprises: ascorbic acid, aspartic acid, cysteine, maleic acid, gentisic acid, glutathione, glutamic acid, mannitol, nicotinamide, calcium chloride, N-t-butyl-alpha-phenylnitrone (PBN), tartaric acid, para-aminobenzoic acid (pABA), chloride ions or salts or combinations thereof; and (iii) a cyclodextrin.

### FIELD OF THE INVENTION

The present invention generally relates to radiopharmaceutical compositions, which comprise a radio-labelled compound and are stabilized with a stabilizer and cyclodextrin as a co-stabilizer. The invention also relates to the use of such radiopharmaceutical compositions in methods of imaging a subject using the radiopharmaceutical compositions. Also described are methods and kits for the preparation of the radiopharmaceutical compositions.

### BACKGROUND TO THE INVENTION

Stabilizers are needed in radiopharmaceutical preparations to reduce the formation of radioimpurities during their shelf life.

Conventional radiopharmaceuticals contain a radiopharmaceutical, a gas, and a formulation that contains a solvent and a stabilizer. Commonly used stabilizers include ethanol, sodium ascorbate, ascorbic acid, maleic acid, gentisic acid and calcium chloride among others.

Cyclodextrins have previously been used to improve the solubility of poorly water soluble substances.

### DESCRIPTION OF THE INVENTION

In one aspect, the present invention provides a radiopharmaceutical composition comprising the following four components: (i) a radio-labelled compound comprising a ¹⁸F-labelled radiopharmaceutical, or a pharmaceutically acceptable salt thereof; (ii) ethanol; (iii) a stabilizer of the radio-labelled compound wherein said stabilizer comprises ascorbic acid; and (iv) a co-stabilizer of the radio-labelled compound wherein said co-stabilizer is a cyclodextrin.

The following subject-matter is provided in combination with the aspect provided above and the additional aspects provided below.

The term radiopharmaceutical has its conventional meaning, and refers to a radioactive compound suitable for in vivo mammalian administration for use in diagnosis or therapy.

The radiopharmaceutical compositions described herein may comprise components as described in US2013129623.

The radio-labelled compound comprises a ¹⁸F-labelled radiopharmaceutical, or a pharmaceutically acceptable salt thereof. Examples of such ¹⁸F-labelled radiopharmaceuticals include [¹⁸F]FDG, [¹⁸F]FMAU, [¹⁸F]FMISO, [¹⁸F]FHBG, [¹⁸F]AV-45, [¹⁸F]AV-19, [¹⁸F]AV-1, [¹⁸F] Flutemetamol, [¹⁸F] Flurpiridaz, [¹⁸F]K5, [¹⁸F]HX4, [¹⁸F]W372, [¹⁸F]VM4-037, [¹⁸F]CP18, [¹⁸F]ML-10, [¹⁸F]T808, [¹⁸F]T807, 2-[¹⁸F]fluoromethyl-L-phenylalanine, or combinations thereof. Preferably, the radio-labelled compound is not [¹⁸F]FLT.

The radio-labelled compound may comprise a compound of Formula (I):

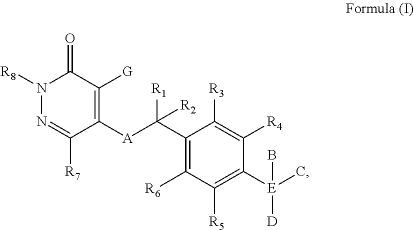

wherein A is selected from N(R⁷), S, O, C(═O), C(═O) O, NHCH₂CH₂O, a bond, or C(═O)N(R⁷);
when present, B is selected from hydrogen, alkoxyalkyl, alkyloxy, aryl, C₁-C₆ alkyl optionally substituted with an imaging moiety, heteroaryl, and an imaging moiety;
when present, C is selected from hydrogen, alkoxyalkyl, alkyloxy, aryl, C₁-C₆ alkyl optionally substituted with an imaging moiety, heteroaryl, and an imaging moiety;
D is selected from hydrogen, alkoxyalkyl, alkyloxy, aryl, C₁-C₆ alkyl optionally substituted with an imaging moiety, heteroaryl, and an imaging moiety; or
C and D, together with the atom to which they are attached, form a three- or four-membered carbocyclic ring;
G is halo or haloalkyl;
n is 0, 1, 2, or 3;
R¹, R², R³, R⁴, R⁵, R⁶ and R⁷ are independently selected from hydrogen, C₁-C₆ alkyl optionally substituted with an imaging moiety, and an imaging moiety;
R⁸ is C₁-C₆ alkyl, optionally substituted with an imaging moiety; and
E is selected from a bond, carbon, and oxygen, provided that when E is a bond, B and C are absent and D is selected from aryl and heteroaryl, and provided that when E is oxygen, B and C are absent and D is selected from hydrogen, alkoxyalkyl, aryl, C₁-C₆ alkyl optionally substituted with an imaging moiety, and heteroaryl;
provided that the imaging moiety comprises ¹⁸F and at least one imaging moiety is present in Formula (I).

Substituent A of Formula (I) may be O. R⁸ may be tert-butyl. G may be chloro.

Compounds of Formula (I) and how to obtain them can be found for example in WO2005079391A2, the contents of which is incorporated herein by reference.

The radio-labelled compound may comprise flurpiridaz, which has the following structure:

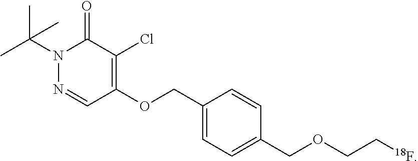

By the term “stabilizer” it is specifically meant radio-stabilizer, which is a compound that inhibits degradation reactions, such as redox processes, by trapping highly-reactive free radicals, such as oxygen-containing free radicals arising from the radiolysis of water. The stabilizers of the invention protect the radio-labelled compound(s) from radiolysis and therefore lower/prevent a drop in the purity of the radio-labelled compound(s) over their shelf life. By the term “co-stabilizer” is meant a compound that enhances the desired effects of the stabilizer.

Radiochemical purity (RCP) is determined using radio TLC or HPLC and can be defined as the ratio of the (radio-labelled) drug substance peak to the total (radio-labelled) peaks in the chromatogram. If one manufactures a radiopharmaceutical with high radioactive concentration (RAC), the drop in RCP during storage is likely to be higher than at lower RAC due to more radiolysis. High radioactivity results in the drug substance destroying itself (i.e. radiolysis). The most efficient stabilizer can be identified by preparing different formulations of radiopharmaceuticals at similar RAC and comparing the drop in RCP over time, typically 8-10 hours for ¹⁸F compounds. The radiopharmaceutical preparation with the smallest drop in RCP during storage has the most effective stabilizer for that specific drug substance.

In some cases, ethanol can be considered to be a stabilizer. Further stabilizers include ascorbic acid, aspartic acid, cysteine, maleic acid, gentisic acid, glutathione, glutamic acid, mannitol, nicotinamide, calcium chloride, N-t-butyl-alpha-phenylnitrone (PBN), tartaric acid and para-aminobenzoic acid (pABA), chloride ions or salts or combinations thereof. The stabilizer of the present invention comprises ascorbic acid. Ethanol may comprise up to 10% (v/v) ethanol in aqueous solution. Preferably, pharmaceutical grade material is used.

The cyclodextrin may comprise: α-cyclodextrin, β-cyclodextrin or γ-cyclodextrin, or pharmaceutically acceptable derivatives or combinations thereof. The cyclodextrin may comprise β-cyclodextrin. The cyclodextrin may comprise hydroxypropyl-beta-cyclodextrin (HPbCD). In the context of the present invention the cyclodextrin is a co-stabilizer.

The radiopharmaceutical composition may comprise a biocompatible carrier. The biocompatible carrier is a fluid, especially a liquid, in which the radiopharmaceutical can be suspended or preferably dissolved, such that the composition is physiologically tolerable, i.e. can be administered to the mammalian body without toxicity or undue discomfort. The biocompatible carrier is suitably an injectable carrier liquid such as sterile, pyrogen-free water for injection; an aqueous solution such as saline (which may advantageously be balanced so that the final product for injection is isotonic); an aqueous buffer solution comprising a biocompatible buffering agent (e.g. phosphate buffer); an aqueous solution of one or more tonicity-adjusting substances (e.g. salts of plasma cations with biocompatible counterions), sugars (e.g. glucose or sucrose), sugar alcohols (e.g. sorbitol or mannitol), glycols (e.g. glycerol), or other non-ionic polyol materials (e.g. polyethyleneglycols, propylene glycols and the like). Preferably, the biocompatible carrier is pyrogen-free water for injection, isotonic saline or phosphate buffer.

The radiopharmaceutical composition may be in a form suitable for mammalian administration. By the phrase “in a form suitable for mammalian administration” it is meant a composition which is sterile, pyrogen-free, lacks compounds which produce toxic or adverse effects, and is formulated at a biocompatible pH (approximately pH 4.0 to 10.5, preferably 4.5 to 9.5, more preferably 4.5 to 7.5 for the agents of the present invention) and physiologically compatible osmolality. Such compositions lack particulates that could risk causing emboli in vivo, and are formulated so that precipitation does not occur on contact with biological fluids (e.g. blood). Such compositions also contain only biologically compatible excipients, and are preferably isotonic.

Preferably, the mammal is an intact mammalian body in vivo, and is more preferably a human subject. Preferably, the radiopharmaceutical can be administered to the mammalian body in a minimally invasive manner, i.e. without a substantial health risk to the mammalian subject even when carried out under professional medical expertise. Such minimally invasive administration is preferably intravenous administration into a peripheral vein of said subject, without the need for local or general anaesthetic.

In a particular embodiment of the present invention, the stabilizer comprises ascorbic acid and ethanol, the cyclodextrin comprises hydroxypropyl-beta-cyclodextrin (HPbCD) and the radio-labelled compound comprises flurpiridaz. The ascorbic acid can be in an amount from about 1 to about 100 mg/mL, the ethanol can be in an amount from about 2 to about 10% (v/v) and the HPbCD can be in an amount of from about 1 to about 100 mg/mL.

In one embodiment of the present invention ascorbic acid is present in an amount from 1 to about 100 mg/mL, for example from about 30 to about 50 mg/mL.

In one embodiment of the present invention HPbCD is present in an amount of from about 1 to about 100 mg/mL, or from about 40 to about 50 mg/mL, for example from about 40 to about 47 mg/ml.

In one embodiment of the present invention ethanol is present in an amount from about 2 to about 10% (v/v), or from about 5 to about 10% (v/v), for example 7% (v/V).

In another aspect, the present invention provides the use of a cyclodextrin as a co-stabilizer in a radiopharmaceutical composition. The definitions of cyclodextrin and radiopharmaceutical composition for this aspect are the same as provided above.

The radiopharmaceutical composition may comprise a radio-labelled compound, wherein the radio-labelled compound is not [¹⁸F]FLT.

The invention also provides a method of imaging a subject using the radiopharmaceutical composition described above.

The invention also provides the radiopharmaceutical composition described above for use in positron emission tomography (PET) imaging.

In another aspect, the present invention provides a method of preparation of a radiopharmaceutical composition comprising combining the following four components: (i) a radio-labelled compound comprising a ¹⁸F-labelled radiopharmaceutical, or a pharmaceutically acceptable salt thereof; (ii) ethanol; (iii) a stabilizer of the radio-labelled compound wherein said stabilizer comprises ascorbic acid; and (iv) a co-stabilizer of the radio-labelled compound wherein said co-stabilizer is a cyclodextrin.

In a further aspect, the present invention provides a kit for the preparation of a radiopharmaceutical composition, comprising: the following four components: (i) a precursor compound for the production of a radio-labelled compound comprising a ¹⁸F-labelled radiopharmaceutical, or a pharmaceutically acceptable salt thereof; (ii) ethanol; (iii) a stabilizer of the radio-labelled compound; and (iv) a co-stabilizer of the radio-labelled compound wherein said co-stabilizer is a cyclodextrin.

A “precursor compound” comprises a non-radioactive derivative of a radiolabelled compound, designed so that chemical reaction with a convenient chemical form of an in vivo-detectable label occurs site-specifically; can be conducted in the minimum number of steps (ideally a single step); and without the need for significant purification (ideally no further purification), to give the desired in vivo imaging agent. Such precursor compounds are synthetic and can conveniently be obtained in good chemical purity. In one embodiment the precursor compound is a non-radioactive derivative of the ¹⁸F-labelled radiopharmaceutical that includes a leaving group, which is replaced with ¹⁸F upon reaction of the precursor compound with a suitable source of ¹⁸F-fluoride.

The term “leaving group” refers to an atom or group of atoms that is displaced as a stable species during a substitution or displacement radiofluorination reaction. Suitable leaving groups include halogens and sulfonate-containing leaving groups. Specific examples of suitable leaving groups include iodide, bromide, chloride, mesylate, triflate, tosylate, nosylate or 1,2-cyclic sulfate.

The term “suitable source of ¹⁸F-fluoride” refers to F-8-fluoride in a chemical form suitable for displacing a leaving group in a nucleophilic substitution reaction to result in the ¹⁸F-labelled radiopharmaceutical. ¹⁸F-fluoride is normally obtained as an aqueous solution from the nuclear reaction ¹⁸O(p,n)¹⁸F and is made reactive by the addition of a cationic counterion and the subsequent removal of water. Suitable cationic counterions should possess sufficient solubility within the anhydrous reaction solvent to maintain the solubility of ¹⁸F. Suitable counterions include large but soft metal ions such as rubidium or caesium, potassium complexed with a cryptand such as Kryptofix™ 222 (K222), or tetraalkylammonium salts. A suitable tetraalkylammonium salt is tetrabutylammonium hydrogen carbonate. A detailed discussion of well-known ¹⁸F labelling techniques can be found in Chapter 6 of the “Handbook of Radiopharmaceuticals” (2003; John Wiley and Sons: M. J. Welch and C. S. Redvanly, Eds.).

Where the ¹⁸F-radiopharmaceutical composes ¹⁸F-flurpiridaz the precursor compound may be the following compound:

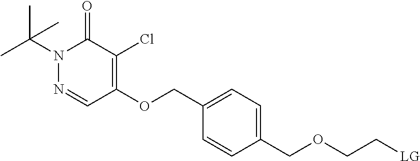

Wherein LG is a leaving group as defined hereinabove. More detail on obtaining this precursor compound, labelling it to obtain ¹⁸F-flurpiridaz, and suitable kit presentations can be found for example in WO2019185932A1 and WO2011097649A2, the contents of which are incorporated herein by reference.

The radiopharmaceutical composition may contain additional optional excipients. For example, such additional optional excipients include: an antimicrobial preservative, pH-adjusting agent, filler, solubiliser or osmolality adjusting agent.

By the term “antimicrobial preservative” is meant an agent which inhibits the growth of potentially harmful micro-organisms such as bacteria, yeasts or moulds. The antimicrobial preservative may also exhibit some bactericidal properties, depending on the dosage employed. The main role of the antimicrobial preservative(s) of the present invention is to inhibit the growth of any such micro-organism in the pharmaceutical composition. The antimicrobial preservative may, however, also optionally be used to inhibit the growth of potentially harmful micro-organisms in one or more components of kits used to prepare said composition prior to administration. Suitable antimicrobial preservative(s) include: the parabens, i.e. methyl, ethyl, propyl or butyl paraben or mixtures thereof; benzyl alcohol; ethanol, phenol; cresol; cetrimide and thiomersal. Preferred antimicrobial preservative(s) are the parabens or ethanol.

The term “pH-adjusting agent” means a compound or mixture of compounds useful to ensure that the pH of the composition is within acceptable limits (approximately pH 4.0 to 10.5, preferably 4.5 to 9.5, more preferably 4.5 to 7.5 for the agents of the present invention) for human or mammalian administration. Suitable such pH-adjusting agents include pharmaceutically acceptable buffers, such as tricine, phosphate, acetate or TRIS [i.e. tris(hydroxymethyl)aminomethane], and pharmaceutically acceptable bases such as sodium carbonate, sodium bicarbonate or mixtures thereof. When the composition is employed in kit form, the pH adjusting agent may optionally be provided in a separate vial or container, so that the user of the kit can adjust the pH as part of a multi-step procedure.

By the term “filler” is meant a pharmaceutically acceptable bulking agent which may facilitate material handling during production and lyophilisation. Suitable fillers include inorganic salts such as sodium chloride, and water soluble sugars or sugar alcohols such as sucrose, maltose, mannitol or trehalose.

By the term “solubiliser” is meant an additive present in the composition which increases the solubility of the radiopharmaceutical in the solvent. A preferred such solvent is aqueous media, and hence the solubiliser preferably improves solubility in water. Suitable such solubilisers include: C₁₋₄ alcohols; glycerine; polyethylene glycol (PEG); propylene glycol; polyoxyethylene sorbitan monooleate; sorbitan monooloeate; polysorbates (e.g. Tween™); poly(oxyethylene) poly(oxypropylene) poly(oxyethylene) block copolymers (Pluronics™); cyclodextrins (e.g. alpha, beta or gamma cyclodextrin, hydroxypropyl-β-cyclodextrin or hydroxypropyl-γ-cyclodextrin) and lecithin.

Preferred solubilisers are cyclodextrins, C₁₋₄ alcohols, polysorbates and Pluronics™, more preferably cyclodextrins and C₂₋₄ alcohols. When the solubiliser is an alcohol, it is preferably ethanol or propanol, more preferably ethanol. Ethanol has potentially several roles, since it can also function as a biocompatible carrier, radioprotectant or antimicrobial preservative. When the solubiliser is a cyclodextrin, it is preferably a gamma cyclodextrin, more preferably hydroxypropyl-β-cyclodextrin (HPbCD). The concentration of cyclodextrin can be from about 0.1 to about 50 mg/ml, preferably between about 5 and about 50 mg/ml, more preferably 25 to 50 mg/ml, most preferably between about 40 and about 50 mg/ml.

The present inventors have found that the use of cyclodextrin as co-stabilizer improves the stability of a radiopharmaceutical compared to using a conventional stabilizer. By adding a cyclodextrin to a radiopharmaceutical formulation, one gets the added value of a more radiochemically stable product, as well as a more physically stable product that is more compatible with plastic contact materials (tubing, sterilising filters, syringes etc.). If the active substance is poorly water soluble, this is even more beneficial.

Calcium disodium edetate may also be used as an additional optional excipient.

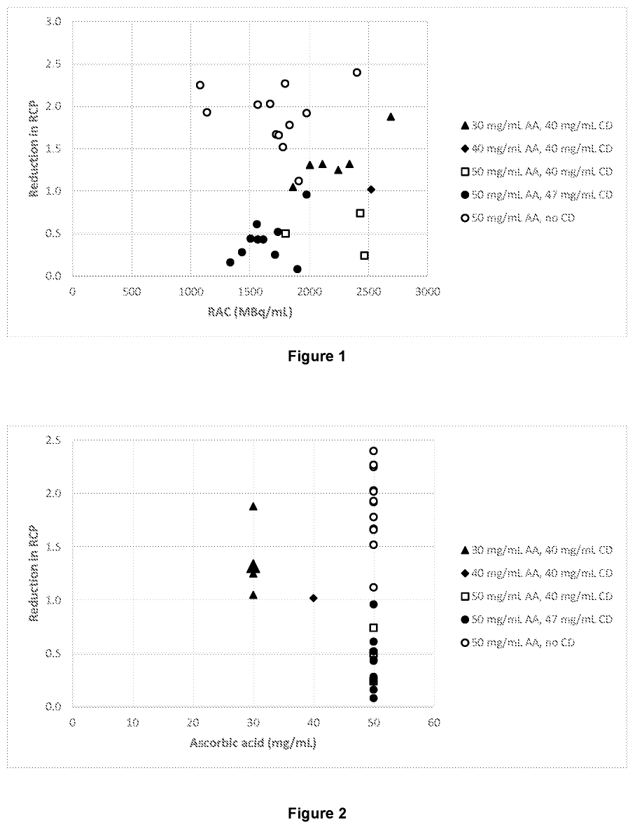

FIG. 1 shows the results of radiochemical purity (RCP) testing of various radiopharmaceutical compositions as a function of radioactive concentration (RAC).

FIG. 2 shows the results of radiochemical purity (RCP) testing of various radiopharmaceutical compositions as a function of ascorbic acid concentration.

As shown in FIG. 1, the RCP is more stable over 10 hours when HPbCD is present in the ascorbic acid formulation than without HPbCD.

The radiostability of [¹⁸F]flurpiridaz is equal or better when prepared with 30 mg/mL ascorbic acid and 40 mg/mL HPbCD than when prepared with 50 mg/mL ascorbic acid without HPbCD. The HPbCD also improves the aqueous solubility of flurpiridaz and reduces the risk of incompatibility with consumer materials (e.g., tubing, sterilising filters, syringes etc.).

As shown in FIG. 2, reduction in RCP is observed over 4 to 10 h when [¹⁸F]flurpiridaz is formulated with 30 to 50 mg/mL ascorbic acid (pH ˜6) with or without 40-47 mg/mL HPbCD. All samples contain ca. 7% (v/v) ethanol and are held under 0-21% (v/v) oxygen headspace gas.

The invention is described with reference to the following non limiting examples.

### Example 1: Radiosynthesis of [¹⁸F]Flurpiridaz with SPE Purification

[¹⁸F]fluoride (ca. 200 GBq) was produced using a GE Medical Systems PETtrace cyclotron with a silver target via the [¹⁸O](p,n) [¹⁸F] nuclear reaction. Total target volumes of 3.2-4.8 mL were used. The radiofluoride was trapped on a Waters QMA cartridge (pre-conditioned with carbonate), and the fluoride was eluted with a solution of tetrabutylammonium hydrogen carbonate (22 mg) in water (100 μL) and acetonitrile (400 μL). Nitrogen was used to drive the solution off the QMA cartridge to the reaction vessel. The [¹⁸F]fluoride was dried for ca. 15 minutes at 110-120° C. under a steady stream of nitrogen and vacuum. The precursor (10.2 mg) in MeCN (1.7 mL) was added to the dried [¹⁸F]fluoride and the reaction mixture was heated at 110 C for 3 minutes. The crude product was then hydrolysed with a solution of NaOH (2 M, 2.3 mL). The hydrolysed crude product was then loaded onto a tC18 SPE cartridge (Waters, product number WAT036800) and purified using the method described below.

The SPE cartridge was washed with ascorbic acid (21 mL) to wash away the acetonitrile, NaOH and hydrophilic chemical and radiochemical impurities. Then the SPE cartridge was washed with a 40% acetonitrile solution in water (11.9 mL) to remove the hydroxy impurity. After this, the first SPE cartridge was connected in series to a second SPE cartridge (Waters, product number WAT036800) and the two were washed in series with 40% acetonitrile solution in water (22.2 mL) followed by a stream of nitrogen to transfer [¹⁸F] Flurpiridaz onto the second cartridge and trap the more lipophilic chemical and radiochemical impurities on the first SPE cartridge. The second SPE cartridge was washed with a 40% acetonitrile solution in water (5.1 mL) followed by ascorbic acid (21 mL) to remove the acetonitrile. The product was then eluted off the second SPE cartridge with a 45% ethanolic solution (9 mL, first 2 mL not collected) to elute [¹⁸F]Flurpiridaz into the product vial.

The first 45 mL product vial was composed of water (42 mL), ethanol (3 mL), calcium disodium edetate (0.25 mg/mL), ascorbic acid (50 mg/mL) and sodium hydroxide (7.5 mg/mL). The second 45 mL product vial was composed of water (42 mL), ethanol (3 mL), calcium disodium edetate (0.25 mg/mL), ascorbic acid (50 mg/mL), hydroxypropyl-beta-cyclodextrin (45 mg/mL; HPbCD) and sodium hydroxide (7.5 mg/mL).

The non-decay corrected yield was 41-44%, resulting in a product with an RAC of ca. 1800 MBq/mL (Table 1). The RCP of the final product was 96-98%.

After 2 hours the RCP decreased by 0.8-1.4% with the formulation vial not containing HPbCD or 0.3-0.4% with the formulation vial containing HPbCD (Table 1). After 4 hours the RCP decreased by 1.1-1.7% with the formulation vial not containing HPbCD or 0.6% with the formulation vial containing HPbCD. As the specification for RCP was 95% at end of shelf-life (8-10 hours), the batch failed when HPbCD was excluded from the formulation.

Furthermore, when HPbCD is used in the formulation, the starting activity can be increased to 350 GBq with a product RAC of ca. 2500 MBq/mL. The RCP is 96-98% with a ca. 0.5-1.3% decrease in RCP over 8-10 hours.

TABLE 1
Representative formulated products of [¹⁸F]Flurpiridaz with and
without hydroxypropyl-beta-cyclodextrin (HPbCD).

| | Starting | RAC | | | | |
|---|---|---|---|---|---|---|
| | activity | (MBq/ | [¹⁸F]fluoride | | RCP | |
| Formulation | (MBq) | mL) | T = 0 h | T = 4 h | T = 0 h | T = 4 h |
| Without HPbCD | 190279 | 1742 | <0.3 | 0.4 | 97.4 | 95.7 |
| Without HPbCD | 200127 | 1911 | 0.3 | 0.5 | 96.1 | 95.0 |
| With HPbCD | 202606 | 1976 | <0.3 | <0.3 | 97.7 | 97.1 |
| With HPbCD | 202902 | 1971 | <0.3 | <0.3 | 97.4 | 96.8 |

As shown above, the present inventors have found that the use of cyclodextrin as co-stabilizer improves the radiostability of a radiopharmaceutical composition compared to using a conventional radiostabilizer alone, e.g. ascorbic acid, or radiostabilizing system, e.g. ascorbic acid and ethanol.

1. A stable radiopharmaceutical composition suitable for mammalian administration comprising:
[¹⁸F] flurpiridaz;
about 30 to about 50 mg/mL ascorbic acid;
about 40 to about 50 mg/mL hydroxypropyl-beta-cyclodextrin (HPbCD); and
ethanol;
wherein the pH of the stable radiopharmaceutical composition is 4.5 to 9.5.

2. A method of imaging a subject in need thereof, comprising:
intravenously administering the stable radiopharmaceutical composition of claim 1; and acquiring an at least one image from the subject after the administration.

3. A method of positron emission tomography (PET) imaging in a subject in need thereof, comprising intravenously administering the radiopharmaceutical composition of claim 1; and acquiring at least one PET image from the subject after the administration.

4. A method of positron emission tomography (PET) imaging in a subject in need thereof, comprising:
intravenously administering a stable radiopharmaceutical composition having a pH of 4.5 to 9.5 and comprising:
[¹⁸F] flurpiridaz:
about 30 to about 50 mg/mL ascorbic acid;
a cyclodextrin;
about 2% to about 10% (v/v) ethanol; and
a biocompatible carrier selected from the group consisting of pyrogen-free water;
isotonic saline and phosphate buffer; and
acquiring at least one PET image from the subject after the administration.
